(19) United States
(12) Patent Application Publication
(10) Pub. No.: US 2017/0115157 A1
(43) Pub. Date: Apr. 27, 2017

(54) MEASURING APPARATUS AND MEASURING METHOD

(71) Applicant: FUJITSU LIMITED, Kawasaki-shi (JP)

(72) Inventors: Yosuke YOKOTA, Setagaya (JP); Musashi TSUDA, Kita (JP); KAZUO IKEMOTO, Kawasaki (JP); Hiroshi Hidaka, Kawasaki (JP); Akira Itasaki, Yokohama (JP); Shinobu KOTANI, Setagaya (JP); Daisuke Endo, Koto (JP); Asako Okazaki, Kawasaki (JP); Kazushi OKADA, Yokohama (JP); TETSUYA SASAKI, Kawasaki (JP); Hirofumi TSUJIMURA, Kawasaki (JP); Wataru HOSHII, Kawasaki (JP)

(73) Assignee: FUJITSU LIMITED, Kawasaki-shi (JP)

(21) Appl. No.: 15/262,141

(22) Filed: Sep. 12, 2016

(30) Foreign Application Priority Data
Oct. 21, 2015 (JP) 2015-207485

(51) Int. Cl. G01G 19/50; A61B 5/107; A61B 5/01; H04N 7/18; A61B 5/00

(52) U.S. Cl. CPC G01G 19/50; H04N 7/183; A61B 5/0077; A61B 5/01; A61B 5/1072

(57) ABSTRACT

A measuring apparatus includes a memory configured to store a program, and a processor configured to execute the program and perform a process including acquiring a first measured value from a first sensor that measures a weight of a subject, judging whether the first measured value satisfies a condition in which a change in the first measured value within a predetermined time is a threshold value or less, acquiring a second measured value from a second sensor that is other than the first sensor and detects biometric information of the subject other than the weight, in a case in which the judging judges that the first measured value satisfies the condition, and recording the first measured value satisfying the condition and the second measured value in correspondence with each other.

### CROSS-REFERENCE TO RELATED APPLICATIONS

This application is based upon and claims the benefit of priority of the prior Japanese Patent Application No. 2015-207485, filed on Oct. 21, 2015, the entire contents of which are incorporated herein by reference.

### FIELD

The embodiments discussed herein are related to a measuring apparatus, a measuring method, and a computer-readable storage medium.

### BACKGROUND

Various measuring apparatuses have been proposed to measure biometric information of a person. For example, Japanese Laid-Open Patent Publication No. 7-303617 proposes a system in which a height or the like of a subject (or person) is input from an input device, and a percent of body fat of the subject based on a weight of the subject measured by a body weight measuring apparatus (or scale) and the height input from the input device. However, since the percent of body fat of the subject is computed based on the measured weight of the subject, the height of the subject that is input, or the like, an accurate percent of body fat cannot be computed unless an accurate height is input. This is because, in a case in which the subject is a child, for example, the child becomes taller as the child grows.

On the other hand, Japanese Laid-Open Patent Publication No. 2010-193977 proposes a method of measuring a mass and a center of gravity of body parts of the subject (or person), based on data acquired by capturing, by a 3-dimensional motion capture camera, the body parts of the subject added with a plurality of markers. However, it requires troublesome and time consuming operations to add the markers on the body parts of the subject. In addition, the subject is required to slowly move the body parts within a capture range, while the 3-dimensional motion capture camera captures the subject. In the case in which the subject is a child, however, it is difficult to make the child move in a predetermined manner, and for this reason, it is difficult to capture an image that is suited for accurately measuring the mass and the center of gravity of the body parts of the child.

Consequently, according to the conventional system and method of measuring the biometric information, it is difficult to accurately measure a plurality of kinds of biometric information.

### SUMMARY

Accordingly, it is an object in one aspect of the embodiments to provide a measuring apparatus, a measuring method, and a computer-readable storage medium, which can accurately measure a plurality of kinds of biometric information.

According to one aspect of the embodiments, a measuring apparatus includes a memory configured to store a program; and a processor configured to execute the program and perform a process including acquiring a first measured value from a first sensor that measures a weight of a subject; judging whether the first measured value satisfies a condition in which a change in the first measured value within a predetermined time is a threshold value or less; acquiring a second measured value from a second sensor that is other than the first sensor and detects biometric information of the subject other than the weight, in a case in which the judging judges that the first measured value satisfies the condition; and recording the first measured value satisfying the condition and the second measured value in correspondence with each other.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a block diagram illustrating an example of a measuring apparatus in a first embodiment;

FIG. 2 is a block diagram illustrating another example of a connection between a computer and sensors;

FIG. 3 is a flow chart for explaining a first example of a measuring process;

FIG. 4 is a flow chart for explaining a second example of the measuring process;

FIG. 5 is a flow chart for explaining a third example of the measuring process;

FIG. 6 is a block diagram illustrating an example of the measuring apparatus in a second embodiment;

FIG. 7 is a disassembled perspective view schematically illustrating the measuring apparatus in the second embodiment;

FIG. 8 is a perspective view illustrating an example of an external appearance of the measuring apparatus in the second embodiment;

FIG. 9 is a flow chart for explaining a fourth example of the measuring process; and

FIG. 10 is a flow chart for explaining a fifth example of the measuring process.

### DESCRIPTION OF EMBODIMENTS

Preferred embodiments of the present invention will be described with reference to the accompanying drawings.

A description will now be given of a measuring apparatus, a measuring method, and a computer-readable storage medium according to illustrative embodiments according to the present invention.

According to one embodiment, in a case in which a change of a measured weight of a subject (or person) within a predetermined time is a threshold value or less, biometric information other than the weight of the subject is detected. The measured weight of the subject and the biometric information detected under a condition in which the change of the measured weight of the subject within the predetermined time is the threshold value or less, are recorded in correspondence with each other.

In this specification, a “subject” refers to a person whose biometric information is to be measured by the measuring apparatus.

FIG. 1 is a block diagram illustrating an example of the measuring apparatus in a first embodiment. A measuring apparatus 1-1 illustrated in FIG. 1 includes a general-purpose computer 2, a force sensor (or body weight measuring apparatus, or scale) 3, a distance measuring sensor 4, a temperature sensor 5, and a camera 6. The computer 2 includes a processor 21, such as a CPU (Central Processing Unit), a storage 22 such as a memory, a display device 23, an input device 24 such as a keyboard, a tag reader, or the like, and an interface (I/F) 25-1 that are connected via a bus 26. The processor 21 executes one or more programs stored in the storage 22 and performs processes such as a measuring process which will be described later. The storage 22 stores, in addition to the one or more programs, various data including parameters used by the one or more programs, intermediate data of computations executed by the processor 21, records of measured values measured for each subject, or the like. The storage 22 may be formed by a non-transitory computer-readable storage medium. The display device 23 may display messages with respect to an operator of the measuring apparatus 1-1, measured results of the subject, or the like. The input device 24 may be operated by the operator, to input commands data, personal information of the subject, or the like to the computer 2.

The force sensor 3 is an example of a first sensor that measures a weight of the subject, as an example of biometric information, by a known method, and outputs a weight value W to the interface 25-1. The distance measuring sensor 4 measures a height of the subject by a known method, and outputs a height value H to the interface 25-1. The temperature sensor 5 measures a body temperature of the subject by a known method, and outputs a body temperature value T to the interface 25-1. The camera 6 captures an image of the subject by a known method, and outputs image data (or video data) of the subject to the interface 25-1. The distance measuring sensor 4, the temperature sensor 5, and the camera 6 are examples of a second sensor that detects biometric information of the subject other than the weight of the subject. The measuring apparatus 1-1 includes at least one second sensor. Accordingly, although 3 second sensors are provided in this example, at least one of the distance measuring sensor 4, the temperature sensor 5, and the camera 6 may be provided. In addition, the second sensor is not limited to the distance measuring sensor 4, the temperature sensor 5, and the camera 6, and may be a sensor that measures biometric information of the subject other than the height, the body temperature, and the image of the subject, such as a foot size of the subject. In other words, the number of second sensors that are provided is not limited to a specific value, and may be one or more. Further, the biometric information of the subject to be measured by the second sensor, other than the weight of the subject, is not limited to specific biometric information.

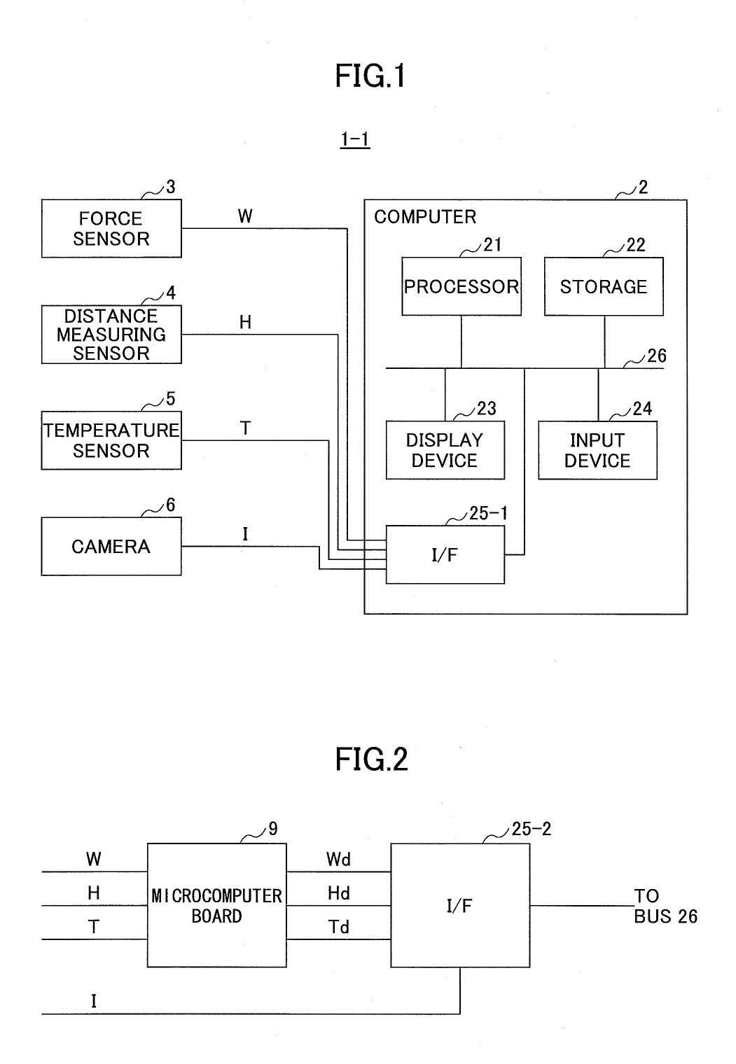

The interface 25-1 illustrated in FIG. 1 includes a function to convert analog values W, H, and T from the sensors 3, 4, and 5 into digital values suited for processing within the computer 2, and inputs the digital values to the processor 21 via the bus 26. The interface 25-1 also includes a function to input digital image data (or digital video data) I from the camera 6 to the processor 21 via the bus 26. In a case in which an interface of the computer 2 has a configuration to input only digital values, a circuit illustrated in FIG. 2 may be used, for example.

FIG. 2 is a block diagram illustrating another example of a connection between the computer and the sensors. A microcomputer board 9 illustrated in FIG. 2 may be provided within the computer 2, or may be externally connected to the computer 2. An interface (I/F) 25-2 is provided in place of the interface 25-1 illustrated in FIG. 1. The microcomputer board 9 is formed by one or a plurality of boards, such as ARDUINO UNO (Registered Trademark) manufactured by Arduino, for example. In this example, the microcomputer board 9 converts the analog values W, H, and T from the sensors 3, 4, and 5 into digital values Wd, Hd, and Td suited for processing within the computer 2, and outputs the digital values Wd, Hd, and Td to the interface 25-2.

On the other hand, the digital image data I from the camera 6 are output directly to the interface 25-2. The interface 25-2 has a function to input the digital values Wd, Hd, and Td and the digital image data I to the processor 21 via the bus 26.

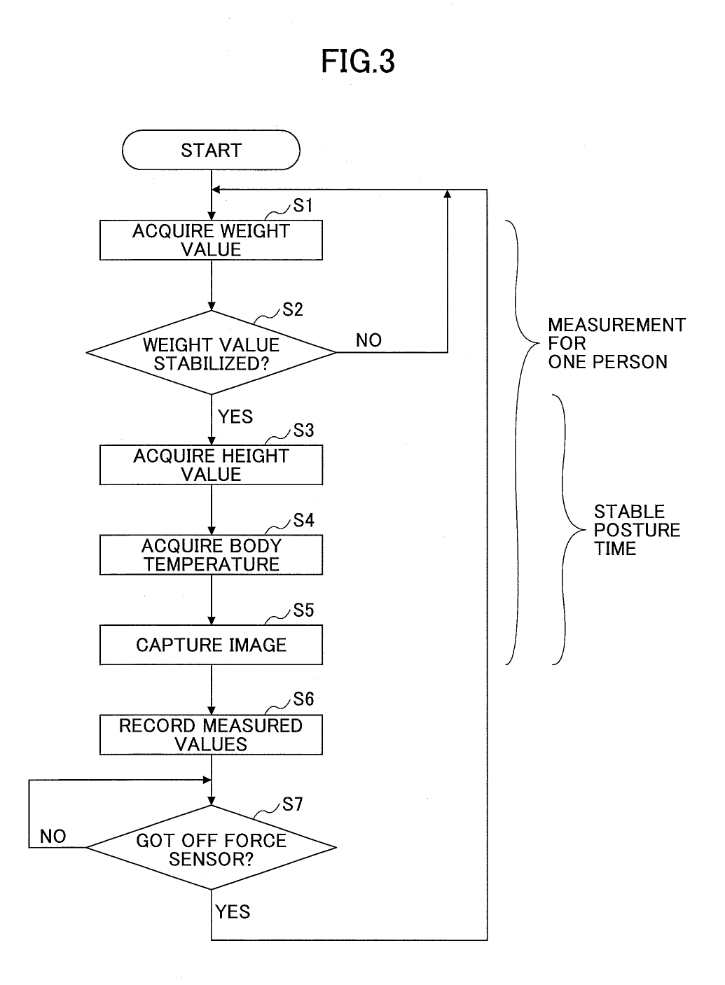

FIG. 3 is a flow chart for explaining a first example of a measuring process. The measuring process may be executed by the computer 2 illustrated in FIG. 1, more particularly, by the processor 21. When a subject stands (or gets) on the force sensor 3 and the measuring process illustrated in FIG. 3 starts, the processor 21, in step S1, acquires the weight value W (or first measured value) from the force sensor 3 that measures the weight of the subject. The processor 21, in step S2, judges whether the weight value W is stabilized. The process returns to step S1 when the judgment result in step S2 is NO, and the process advances to step S3 which will be described later when the judgment result in step S2 is YES.

The processor 21, in step S2, may judge whether the weight value W is stabilized, in the following manner, for example. First, by waiting until the weight value W of the subject becomes a predetermined value or greater, it is possible to judge that the subject got on the force sensor 3. Then, in a case in which a mean deviation of the weight value W within the predetermined time, after the weight value W becomes a predetermined value or greater, is less than a threshold value, it is possible to judge that the weight value W is stabilized. For example, in the case in which the subject is a child, the processor 21 waits until the weight value W of the child becomes the predetermined value (for example, 5 kg) or greater, and if the mean deviation of the weight value W within the predetermined time (for example, 2 seconds) is less than the threshold value (for example, 1 kg), the processor 21 may judge that the weight value W of the child stabilized. Of course, other methods may be employed to judge whether the weight value W is stabilized. The method may judge that the weight value W is stabilized, when a condition in which a change in the weight value W within the predetermined time is the threshold value or less, is satisfied.

The processor 21, in step S3 acquires the height value H (or second measured value) from the distance measuring sensor 4 that measures the height of the subject. The processor 21, in step S4, acquires the body temperature value T (or second measured value) from the temperature sensor 5 that measures the body temperature of the subject. In addition, the process 21, in step S5, acquires the image data I (or second measured value) from the camera 6 that captures the image of the subject. The image data I may indicate a still image or a dynamic image of the subject in its entirety (that is, entire body or full-length image of the subject) or a part of the subject (for example, face of the subject). Steps S3 through S5 simply need to be triggered and performed when the judgment result in step S2 becomes YES, and the order in which step S3, S4, and S5 are performed is not limited to a specific order. In addition, 2 or more steps amongst steps S3, S4, and S5 may be performed in parallel (or simultaneously).

Accordingly, the measuring process with respect to one subject is executed by steps S1 through S5. In addition, in steps S3 through S5 after the judgment result in step S2 becomes YES, the weight value W of the subject is stabilized, and as will be described later, it may be assumed that the subject is standing at attention and the posture of the subject is upright. Hence, while steps S3 through S5 are performed, the subject is in a stable posture time (or time period) in which the posture of the subject is stable. The biometric information other than the weight value W of the subject, such as the height value H, the body temperature value T, and the image data I, can be obtained within the stable posture time. As a result, a plurality of kinds of biometric information can be measured accurately, without requiring awareness by the subject, and without applying load on the subject. Furthermore, the stable posture time may be set to a relatively short time of approximately 1 second, for example.

The processor 21, in step S6, records the weight value W of the subject judged to have stabilized (that is, at a point in time when the judgment result in step S2 becomes YES), in correspondence with the height value H, the body temperature value T, and the image data I of the same subject acquired in steps S3 through S5, in the storage 22. A recording format of the biometric information, such as the weight value W, the height value H, the body temperature value T, and the image data I, that are recorded in the storage 22 for each subject (or person), is not limited to a specific format. A clock function of the processor 21 may be used to add time information, such as a date and time when the biometric information is measured, to the biometric information that is recorded in the storage 22.

The processor 21, in step S7, judges whether the subject got off the force sensor 3. When the judgment result in step S7 is YES, the process returns to step S1, to start the measuring process with respect to the next subject (or person). The processor 21 may judge that the subject got off the force sensor 3 when the weight value W of the subject is less than the predetermined value. For example, in the case in which the subject is a child, the processor 21 waits until the weight value W of the child becomes less than the predetermined value (for example, 5 kg), and if the weight value W of the child becomes less than the predetermined value, the processor 21 may judge that the child got off the force sensor 3. Of course, other methods may be employed to judge whether the subject got off the force sensor 3.

In this example, the processor 21 may execute the program stored in the storage 22 to perform a process including:

acquiring a first measured value from a first sensor (for example, the force sensor 3) that measures a weight of a subject;

judging whether the first measured value stabilized and satisfies a condition in which a change in the first measured value within a predetermined time is a threshold value or less;

acquiring a second measured value from a second sensor (for example, at least one of the distance measuring sensor 4, the temperature sensor 5, and the camera 6) that is other than the first sensor and detects biometric information of the subject other than the weight, in a case in which the judging judges that the first measured value satisfies the condition; and

recording the first measured value satisfying the condition and the second measured value in correspondence with each other in the storage 22.

In general, when the subject measures his/her own weight, the subject in many cases gets on the force sensor (or body weight measuring apparatus, or scale) 3 and stands at attention in the upright posture. Similarly, in the case in which the subject is a child, the child in many cases also gets on the force sensor 3 and stands at attention in the upright posture. For this reason, in the state in which the subject is on the force sensor 3 and the measured weight value W is stabilized, it may be assumed that the subject is standing at attention in the upright posture. This state is suited for accurately measuring one or a plurality of biometric information other than the weight of the subject. When the subject is standing at attention in the upright posture, it is easier to accurately measure the height value H of the subject by the distance measuring sensor 4. In addition, it is easier to identify exposed skin parts of the subject, such as a face of the subject, and accurately measure the body temperature value T of the subject by the temperature sensor 5. Moreover, when the subject is standing at attention in the upright posture, it is easier to identify the entire body, the face position, or the like of the subject, for example, and accurately capture the entire body, the face, or the like of the subject by the camera 6.

Therefore, in this embodiment, in a case in which it is judged that the measured weight value W stabilized, this stabilization of the measured weight value W is used as a trigger for measuring the biometric information other than the weight of the subject. As described above, in this example, the biometric information other than the weight of the subject includes the height value H, the body temperature T, and the image data I. Hence, it is possible to accurately measure and record the height value H, the body temperature T, and the image data I of the subject at a point in time when the stable and accurate weight value W is measured, without requiring time consuming and troublesome operations to be performed. Particularly in the case in which the subject is a child, it is difficult to keep the subject still while separately measuring the various kinds of biometric information. However, this embodiment utilizes the fact that the subject in many cases gets on the force sensor 3 and stands at attention in the upright posture, to accurately measure the weight and one or more kinds of biometric information other than the weight in a simple manner, and collectively record the weight and the one or more kinds of biometric information other than the weight. Accordingly, the biometric information such as the weight, height, body temperature, and image data of the subject can be recorded for health care of the subject, for example.

In a case in which the subject is not standing at attention in the upright posture, the processor 21 may urge the measuring process to be executed again. For example, in a case in which a divergence between the height value H of the subject acquired in step S3 illustrated in FIG. 3 and the recorded height value of the same subject is large, such that the divergence of the height value is unlikely to occur in a human being, or a divergence between the body temperature value T of the subject acquired in step S4 illustrated in FIG. 3 and the recorded body temperature value of the same subject is large such that the divergence of the body temperature value is unlikely to occur in the human being, the measuring process may be executed again. More particularly, first step may be provided between steps S3 and S4, to judge whether the divergence of the height value is a threshold value or greater. When the judgment result in first step is NO, the process may advance to step S4. On the other hand, when the judgment result in first step is YES, second step may be provided to urge the measuring process to be executed again, and the process after second step may return to step S1. Third step may be provided between steps S4 and S5, to judge whether the divergence of the body temperature value is a threshold value or greater. When the judgment result in third step is NO, the process may advance to step S5. On the other hand, when the judgment result in third step is YES, fourth step may be provided to urge the measuring process to be executed again, and the process after fourth step may return to step S1. In this case, it is possible to further improve the measuring accuracy.

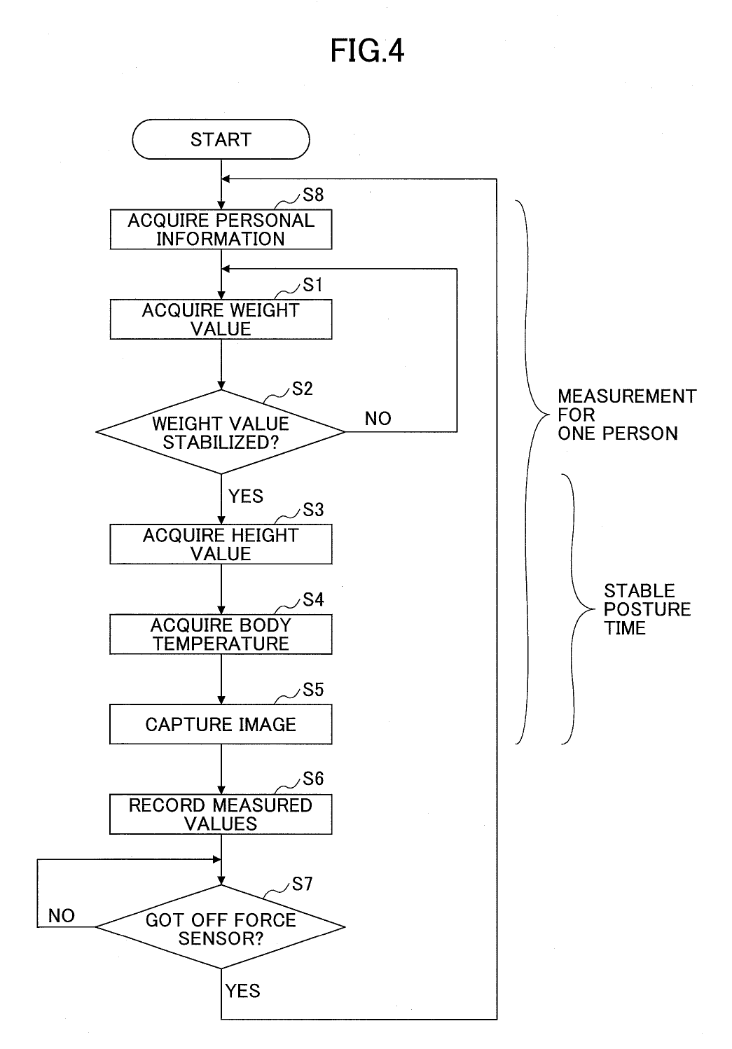

FIG. 4 is a flow chart for explaining a second example of the measuring process. In FIG. 4, those steps that are the same as those corresponding steps in FIG. 3 are designated by the same reference numerals, and a description thereof will be omitted. In FIG. 4, the processor 21, in step S8, acquires personal information of the subject input from the input device 24. In a case in which the input device 24 is the keyboard, the operator may input the personal information of the subject from the keyboard to the processor 21. In a case in which the input device 24 is the tag reader, the personal information of the subject stored in a tag (for example, RFID (Radio Frequency IDentification) tag) carried by the subject may be read by the tag reader and input to the processor 21. In this example, the processor 21 may execute the program stored in the storage 22 to perform the process further including acquiring the personal information of the subject from the input device 24. Each subject can be identified by the personal information. Hence, the processor 21, in step S6, can record the weight value W, the height value H, the body temperature value T, and the image data I, in correspondence with the acquired personal information of the subject, in the storage 22, for example.

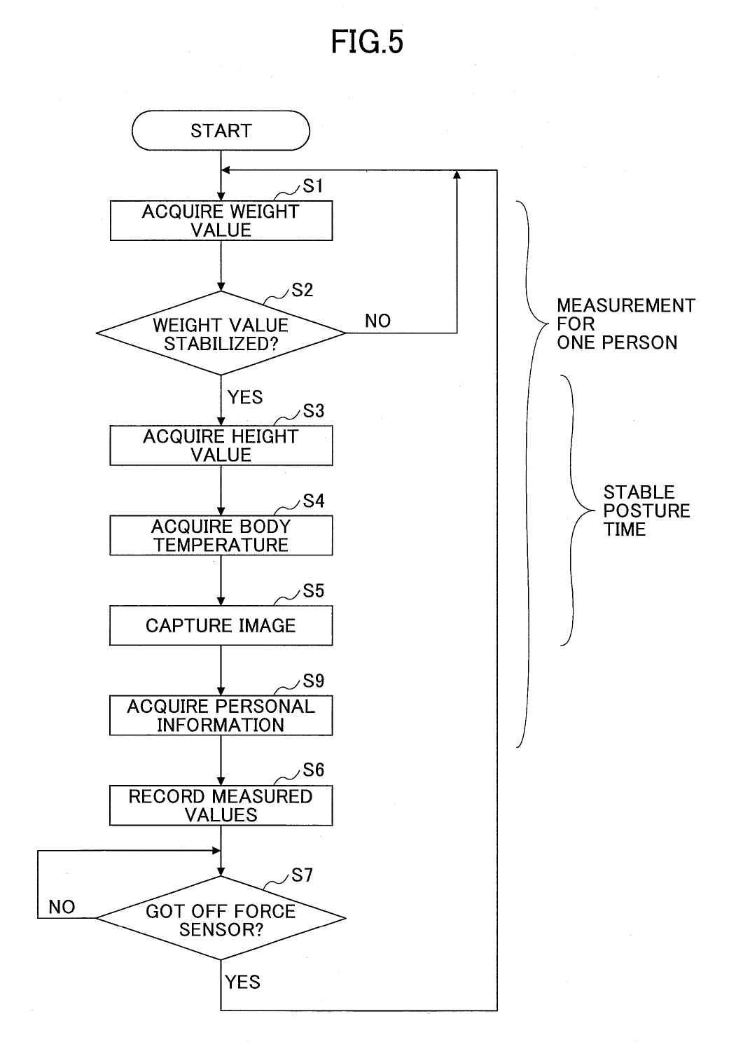

FIG. 5 is a flow chart for explaining a third example of the measuring process. In FIG. 5, those steps that are the same as those corresponding steps in FIG. 3 are designated by the same reference numerals, and a description thereof will be omitted. In FIG. 5, the processor 21, in step S9, extracts input face data from the image data I of the subject input from the camera 6, and performs a known face recognition to match the input face data to registered face data of each subject prestored in the storage 22, and to acquire the personal information of the subject prestored in the storage 22 with respect to the registered face data matching the input face data. In this example, the processor 21 may execute the program stored in the storage 22 to perform the process further including acquiring the personal information of the subject captured by the camera 6 by performing the face recognition of the subject. Each subject can be identified by the personal information. Hence, the processor 21, in step S6, can record the weight value W, the height value H, the body temperature value T, and the image data I, in correspondence with the acquired personal information of the subject, in the storage 22, for example.

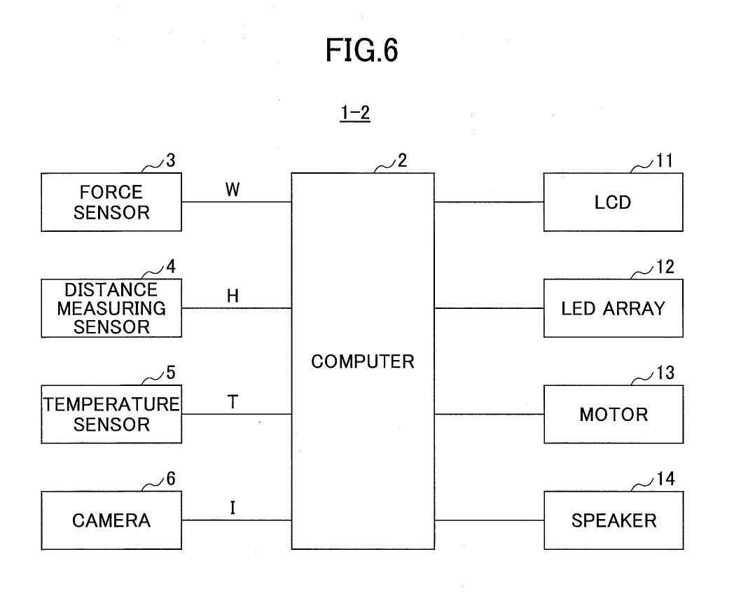

FIG. 6 is a block diagram illustrating an example of the measuring apparatus in a second embodiment. In FIG. 6, those parts that are the same as those corresponding parts in FIG. 1 are designated by the same reference numerals, and a description thereof will be omitted. In FIG. 6, the configuration of the computer 2 is the same as the configuration of the computer 2 illustrated in FIG. 1. A measuring apparatus 1-2 illustrated in FIG. 6 further includes an LCD (Liquid Crystal Display) 11, an LED (Light Emitting Diode) array 12, a motor 13, and a speaker 14 that connect to the interface 25-1 of the computer 2.

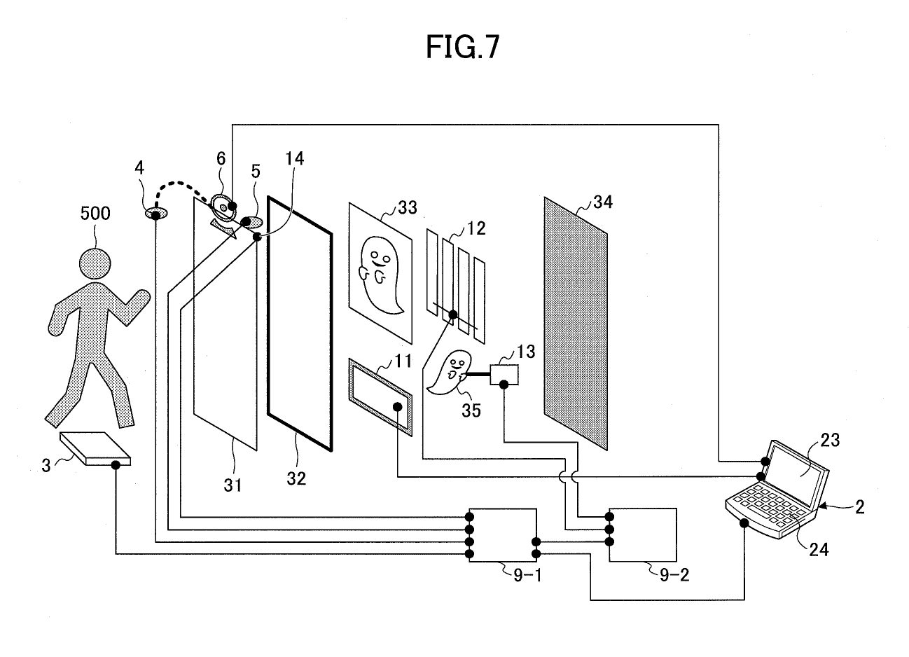

FIG. 7 is a disassembled perspective view schematically illustrating the measuring apparatus in the second embodiment. In FIG. 7, those parts that are the same as those corresponding parts in FIG. 6 are designated by the same reference numerals, and a description thereof will be omitted. In FIG. 7, microcomputer boards 9-1 and 9-2 correspond to the microcomputer board 9 illustrated in FIG. 2, for example. In addition, the computer 2 may be formed by a PC (Personal Computer) that includes the interface 25-2 illustrated in FIG. 2, for example. In this example, the camera 6 and the interface 25-2 (not illustrated) of the computer 2 may be connected by an USB (Universal Serial Bus) cable, for example. In addition, the microcomputer board 9-1 and the interface 25-2 of the computer 2 may be connected by an USB cable, for example. Further, the LCD 11 and the interface 25-2 of the computer 2 may be connected by a VGA (Video Graphic Array) cable, for example. A general signal line, for example, may connect the microcomputer board 9-1 to the force sensor 3, the distance measuring sensor 4, the temperature sensor 5, and the speaker 14. A general signal line, for example, may connect the microcomputer board 9-1 to the LED array 12 and the motor 13. A general signal line may connect the microcomputer board 9-1 and the microcomputer board 9-2.

In FIG. 7, the force sensor 3 is arranged at a position where a subject (or person) 500 standing on the force sensor 3 is reflected in a one-way mirror (or two-way mirror) film 31. The distance measuring sensor 4, the temperature sensor 5, the camera 6, and the speaker 14 are arranged in a periphery of the one-way mirror film 31. The one-way mirror film 31 is provided on a surface of a transparent acrylic plate 32. The LCD 11, a projection film 33, and the LED array 12, which is an example of the light source, are arranged between the acrylic plate 32 and a light blocking black curtain 34. A cutout shape (for example, an arbitrary shape including the shape of a character, such as a ghost) made of paper material or the like and capable of blocking light is formed on the projection film 33. When the LED array 12 is turned on and emits light, the cutout shape of the ghost from the projection film 33 appears through the one-way mirror film 31 as a shadow picture to the subject 500. On the other hand, when the LED array 12 is turned off and emits no light, the inside of a measuring apparatus 1-2 becomes darker than the outside of the measuring apparatus 1-2. In this latter case, the cutout shape of the ghost from the projection film 33 does not appear through the one-way mirror film 31 as a shadow picture to the subject 500, and instead, the image of the subject 500 is reflected in the one-way mirror film 31.

In addition, a model 35 of a ghost is driven by the motor 13, and may move in and out of a housing (not illustrated) of the measuring apparatus 1-2, for example. The model 35 may move to guide the subject 500 onto the force sensor 3, urge the subject 500 to remain still, notify the subject 500 of the start and end of the measuring process, or the like. The manner in which the model 35 moves is not limited to certain movement patterns. A position where the model 35 is arranged is not limited to a specific position, as long as the model 35 does not interfere with the cutout shape of the ghost from the projection film 33 appearing through the one-way mirror film 31 as the shadow picture to the subject 500 while the LED array 12 is turned on and emits light. The model 35 may be arranged inside the housing of the measuring apparatus 1-2, or may be arranged outside the housing of the measuring apparatus 1-2. For example, the model 35 may be provided at a position inside the housing closer to the black curtain 34 than to the LED array 12, or at a position outside the housing on a back side of the housing, that is, on a side of the housing opposite from the one-way mirror film 31.

In this example, the LCD 11 includes a backlight (not illustrated). Accordingly, the subject 500 can see the display on the LCD 11 through the one-way mirror film 31.

Because the one-way mirror film 31 is provided, when the LED array 12 is turned on and emits light, the subject 500 standing on the force sensor 3 can see the shadow picture of the ghost projected on the projection film 33 through the one-way mirror film 31. On the other hand, when the LED array 12 is turned off and emits no light, the subject 500 standing on the force sensor 3 can see his/her own image reflected in the one-way mirror film 31, but cannot see the shadow picture of the ghost through the one-way mirror film 31.

The arrangement of the projection film 33 and the LED array 12 provided between the acrylic plate 32 and the light blocking black curtain 34 is not limited to the arrangement schematically illustrated in FIG. 7.

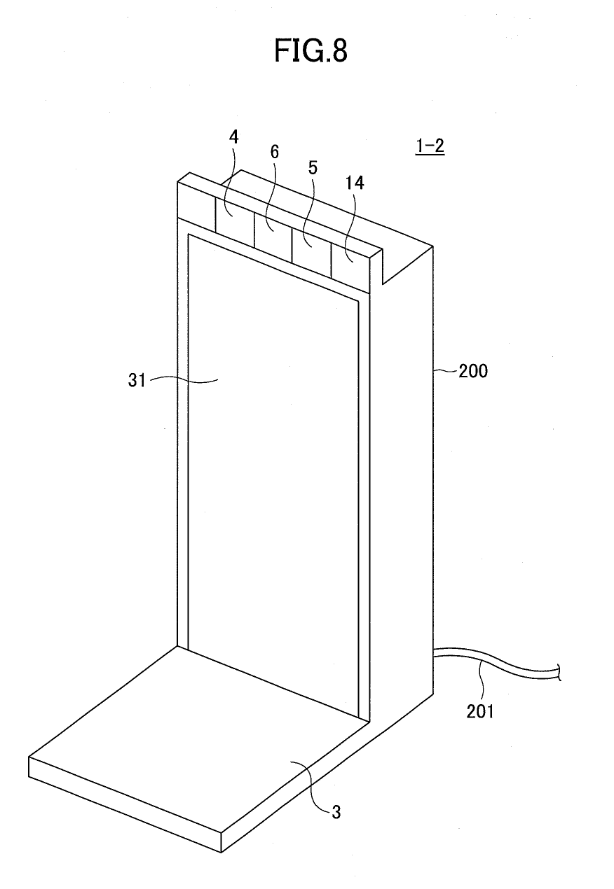

FIG. 8 is a perspective view illustrating an example of an external appearance of the measuring apparatus in the second embodiment. In FIG. 8, those parts that are the same as those corresponding parts in FIG. 7 are designated by the same reference numerals, and a description thereof will be omitted. In the example illustrated in FIG. 8, in order to obtain properties of the one-way mirror film 31 which appears as a mirror film when viewed from a brighter side and appears as a transmission film when viewed from a darker side, a region from the one-way mirror film 31 to the black curtain 34 is covered by a housing 200. In addition, the housing 200 provides coverage so that light may be input thereto and light may be output therefrom, only through the one-way mirror film 31. In other words, the acrylic plate 32, the projection film 33, the LCD 11, the LED array 12, the motor 13, and the black curtain 34 are accommodated within the housing 200. In this example, the distance measuring sensor 4, the temperature sensor 5, the camera 6, and the speaker 14 are arranged at positions in an upper part of the housing 200. However, the positions of the distance measuring sensor 4, the temperature sensor 5, and the camera 6 are not limited the upper part of the housing 200, and these sensors may be arranged at any position capable of detecting the biometric information of the subject 500. In addition, the position of the speaker 14 is not limited to the upper part of the housing 200, and the speaker 14 may be arranged at any position enabling the subject 500 to easily hear audio information (or speech), warning sounds, or the like. In this example, the measuring apparatus 1-2 is connected to the computer 2 and the microcomputer board 9-1 by a cable 201. The force sensor 3 and the housing 200 are integrally provided in this example, however, the force sensor 3 and the housing 200 may be separate bodies.

Because the one-way mirror film 31 functions as a mirror when the LED array 12 is turned off, the subject 500 standing on the force sensor 3 can see his/her own image reflected in the one-way mirror film 31. In addition, when the LED array 12 is turned on, the subject 500 standing on the force sensor 3 can see the shadow picture of the ghost projected on the projection film 33 (hereinafter also referred to as “projected image”) through the one-way mirror film 31. When the subject 500 pays attention to the projected image, the subject 500 is likely to stand at attention in the upright posture. On the other hand, the subject 500 standing on the force sensor 3 can see the display on the LCD 11 through the one-way mirror film 31. When the subject 500 pays attention to the display on the LCD 11 through the one-way mirror film 31, the subject 500 is likely to stand at attention in the upright posture. Particularly in a case in which the projected image and/or the display on the LCD 11 includes information urging the subject 500 to remain still, the subject 500 standing on the force sensor 3 is likely to stand at attention in the upright posture. Further, in a case in which the audio information, the warning sound, or the like output from the speaker 14 urges the subject 500 to remain still, the subject 500 standing on the force sensor 3 is likely to stand at attention in the upright posture.

Moreover, the LED array 12 may be turned on to indicate the start of the measuring process or to urge the subject 500 to remain still, and the LED array 12 may be turned off to indicate the end of the measuring process. Similarly, audio information, warning sound, or the like indicating the start or end of the measuring process may be output from the speaker 14. In addition, audio information, warning sound, or the like urging the subject 500 to remain still may be output from the speaker 14.

The model 35 may be formed to an arbitrary shape, character, or the like, and the shape of the model 35 is not limited to a specific shape. In a case in which the image of the ghost is displayed on the LCD 11, the motor 13, the model 35, and the projection film 33 may be omitted.

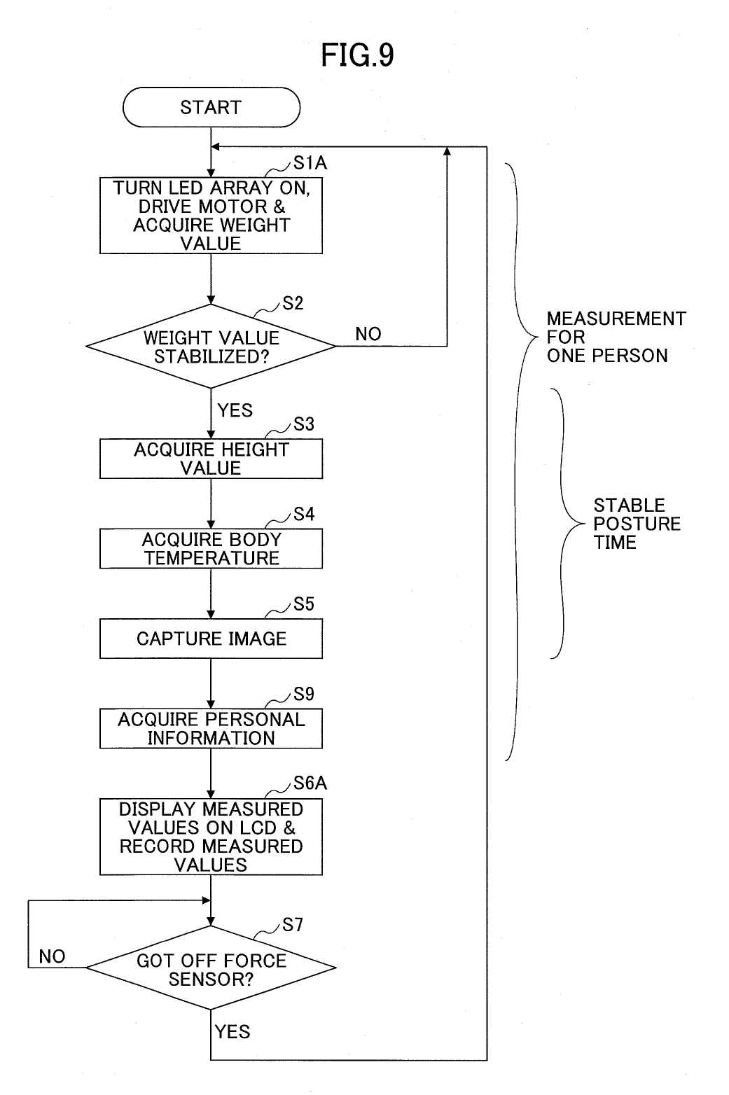

FIG. 9 is a flow chart for explaining a fourth example of the measuring process. In FIG. 9, those steps that are the same as those corresponding steps in FIG. 5 are designated by the same reference numerals, and a description thereof will be omitted. In FIG. 9, the processor 21 of the computer 2 illustrated in FIG. 6, in step S1A, turns on the LED array 12 via the microcomputer boards 9-1 and 9-2 to emit light and project the shadow picture of the ghost through the projection film 33. The processor 21, in step S1A, also starts driving the motor 13 via the microcomputer boards 9-1 and 9-2 to move the model 35 outside the housing 200 of the measuring apparatus 1-2, for example, and urge the subject 500 to remain still. The processor 21, in step S1A, further acquires, via the microcomputer board 9-1, the weight value W from the force sensor 3 that measures the weight of the subject 500 standing on the force sensor 3. In this case, it is possible to notify the subject 500 of the start of the measuring process by turning on the LED array 12, for example. It is also possible to notify the subject 500 of the start of the measuring process by projecting the shadow picture of the ghost on the projection film 33, or by moving the model 35 outside the housing 200 of the measuring apparatus 1-2. A display urging the subject 500 to remain still until the weight value W stabilizes may be displayed on the LCD 11. The process 21, in step S6A, displays on the LCD 11 the weight value W of the subject 500 judged to have stabilized (that is, at a point in time when the judgment result in step S2 becomes YES), in correspondence with the height value H, the body temperature value T, and the image data I of the same subject 500 acquired in steps S3 through S5, and records the measured biometric information in the storage 22. The LCD 11 may display, together with the measured biometric information, the recorded biometric information of the same subject 500 that is most recently recorded, for example. The LCD 11 may also display the measured biometric information and the recorded biometric information measured in the past of the same subject 500, in a graph format. By displaying on the LCD 11 the measured biometric information and the recorded biometric information measured in the past of the same subject 500, it is possible to know a change in the biometric information of the subject 500. In the case in which the subject 500 is a child, it is possible to know a rate of growth of the child. After a predetermined time elapses from the time when the biometric information is displayed on the LCD 11, or when the judgment result in step S7 becomes YES, the processor 21 may turn off the LED array 12 and stop driving the motor 13, via the microcomputer boards 9-1 and 9-2, and end the display of the measured biometric information on the LCD 11. In this case, the turning off of the LED array 12 may notify the subject 500 of the end of the measuring process. Alternatively, the shadow picture of the ghost no longer being projected on the projection film 33 may notify the subject 500 of the end of the measuring process.

After a predetermined time elapses from the time when the biometric information is displayed on the LCD 11, or when the judgment result in step S7 becomes YES, the processor 21 may display a message “good bye” on the LCD 11 in place of the measured biometric information, to notify the subject 500 of the end of the measuring process. In this case, the processor 21 may thereafter turn off the LED array 12 and stop driving the motor 13, via the microcomputer boards 9-1 and 9-2, and end the display of the message on the LCD 11.

In this example, the LCD 11 may form an example of a display device configured to display the first measured value (that is, the weight value W) and the second measured value (that is, at least one of the height value H, the body temperature value T, and the image data I). The display device in this example may display the first and second measured values that are measured, together with the recorded first and second measured values that are measured and recorded in the past. The display device in this example may further display a difference between the first and second measured values that are measured, and the recorded first and second measured values that are measured and recorded in the past. Of course, the display device is not limited to the LCD 11, and may be formed by a display device of a portable terminal (not illustrated) communicable with the measuring apparatus 1-2, for example. For example, the portable terminal may include a smartphone, a tablet, or the like. Moreover, the display device in this example may display the first and second measured values that are measured, the recorded first and second measured values that are measured and recorded in the past, or the like, on both the LCD 11 and the display device of the portable terminal.

Steps S1A and S6A illustrated in FIG. 9 may be performed in place of steps S1 and S6 illustrated in FIG. 3 or FIG. 4.

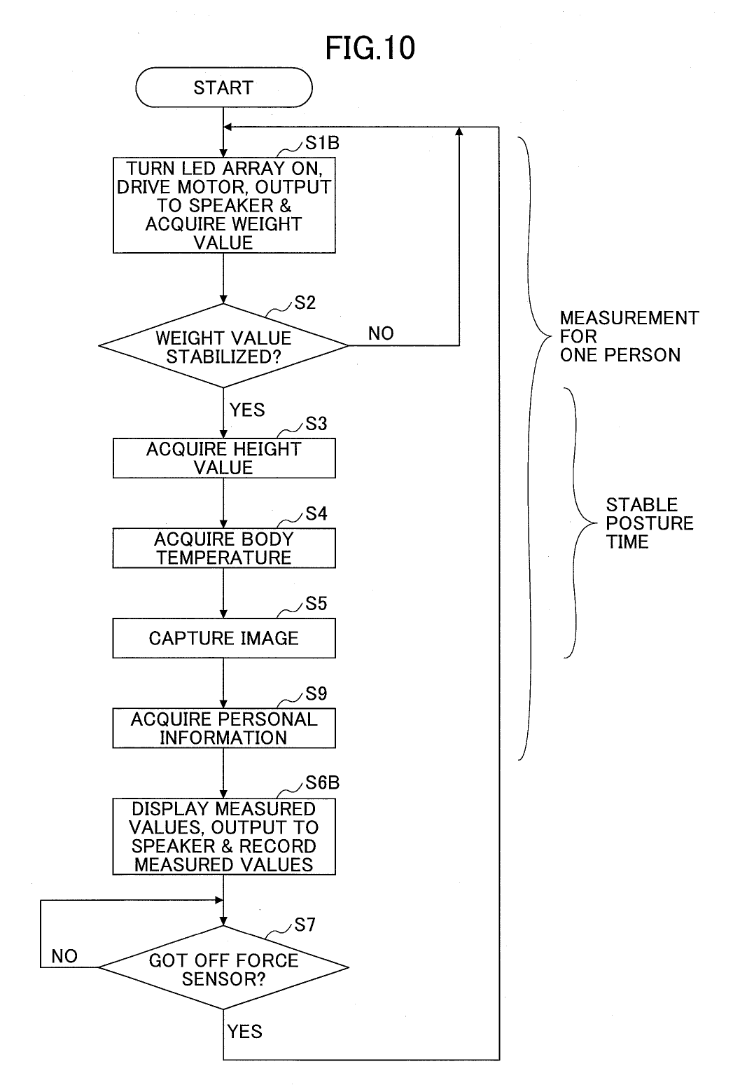

FIG. 10 is a flow chart for explaining a fifth example of the measuring process. In FIG. 10, those steps that are the same as those corresponding steps in FIG. 5 are designated by the same reference numerals, and a description thereof will be omitted. In FIG. 10, the processor 21 of the computer 2 illustrated in FIG. 6, in step S1B, turns on the LED array 12 via the microcomputer boards 9-1 and 9-2 to emit light and project the shadow picture of the ghost through the projection film 33. The processor 21, in step S1B, also starts driving the motor 13 via the microcomputer boards 9-1 and 9-2 to move the model 35 outside the housing 200 of the measuring apparatus 1-2, for example, and urge the subject 500 to remain still. The processor 21, in step S1B, further outputs audio information, warning sound, or the like from the speaker 14, via the microcomputer 9-1, to urge the subject 500 to remain still. For example, the audio information output from the speaker 14 may be a message “please remain still”. The processor 21, in step S1B, also acquires, via the microcomputer board 9-1, the weight value W from the force sensor 3 that measures the weight of the subject 500 standing on the force sensor 3. In this case, it is possible to notify the subject 500 of the start of the measuring process by turning on the LED array 12, projecting the shadow picture of the ghost on the projection film 33, and moving the model 35 outside the housing 200 of the measuring apparatus 1-2. In addition, a display urging the subject 500 to remain still until the weight value W stabilizes may be displayed on the LCD 11, or audio information urging the subject 500 to remain still until the weight value W stabilizes may be output from the speaker 14. The processor 21, in step S6B, displays on the LCD 11 the weight value W of the subject 500 judged to have stabilized (that is, at a point in time when the judgment result in step S2 becomes YES), in correspondence with the height value H, the body temperature value T, and the image data I of the same subject 500 acquired in steps S3 through S5, and records the measured biometric information in the storage 22. In addition, the processor 21, in step S6B, outputs from the speaker 14, via the microcomputer board 9-1, audio information, warning source, or the like notifying the subject 500 of the end of the measuring process, such as a voice message “good bye”. After a predetermined time elapses from the time when the biometric information is displayed on the LCD 11, or when the judgment result in step S7 becomes YES, the processor 21 may turn off the LED array 12 and stop driving the motor 13, via the microcomputer boards 9-1 and 9-2, to end the display of the measured biometric information on the LCD 11, and end the output from the speaker 14 via the microcomputer board 9-1. In this case, the turning off of the LED array 12 may notify the subject 500 of the end of the measuring process. Alternatively, the shadow picture of the ghost no longer being projected on the projection film 33 may notify the subject 500 of the end of the measuring process.

Steps S1B and S6B illustrated in FIG. 10 may be performed in place of steps S1 and S6 illustrated in FIG. 3 or FIG. 4.

Similarly as in the case of the fourth example of the measuring process described above, the LCD 11 may form an example of the display device configured to display the first and second measured values that are measured, together with the recorded first and second measured values that are measured and recorded in the past, or display a difference between the first and second measured values that are measured, and the recorded first and second measured values that are measured and recorded in the past. Of course, the display device is not limited to the LCD 11, and may be formed by a display device of a portable terminal (not illustrated) communicable with the measuring apparatus 1-2, for example.

The shadow picture projected on the projection film 33 may include a message. Similarly, the model 35 may include a message.

In FIG. 7, the LCD 11, the projection film 33, the black curtain 34, the motor 13, and the model 35 may be omitted, and a mirror may be provided in place of the one-way mirror film 31 and the acrylic plate 32. In this case, the subject 500 standing on the force sensor 3 can see his/her own image reflected in the mirror, and is thus likely to stand at attention in the upright posture. Of course, a wall having a picture drawn thereon and/or a message written thereon may be provided in place of the one-way mirror film 31 and the acrylic plate 32. In this case, the subject 500 standing on the force sensor 3 may pay attention to the drawn picture and/or the written message on the walk, and is thus likely to stand at attention in the upright posture.

According to the embodiments described above, it is possible to accurately measure a plurality of kinds of biometric information.

The description above use terms such as “identify”, or the like to describe the embodiments, however, such terms are abstractions of the actual operations that are performed. Hence, the actual operations that correspond to such terms may vary depending on the implementation, as is obvious to those skilled in the art.

Although the embodiments and examples are numbered with, for example, “first,” “second,” etc., the ordinal numbers do not imply priorities of the embodiments or examples. Many other variations and modifications will be apparent to those skilled in the art.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A measuring apparatus comprising:
a memory configured to store a program; and
a processor configured to execute the program and perform a process including
acquiring a first measured value from a first sensor that measures a weight of a subject;
judging whether the first measured value satisfies a condition in which a change in the first measured value within a predetermined time is a threshold value or less;
acquiring a second measured value from a second sensor that is other than the first sensor and detects biometric information of the subject other than the weight, in a case in which the judging judges that the first measured value satisfies the condition; and
recording the first measured value satisfying the condition and the second measured value in correspondence with each other.

2. The measuring apparatus as claimed in claim 1, wherein the second sensor includes at least one of a distance measuring sensor that measures a height of the subject, a temperature sensor that measures a body temperature of the subject, and a camera that captures an image of the subject.

3. The measuring apparatus as claimed in claim 1, wherein the processor performs the process further including
acquiring personal information of the subject,
wherein the recording records the first measured value and the second measured value in correspondence with the personal information.

4. The measuring apparatus as claimed in claim 3, wherein
the second sensor includes a camera that captures an image of the subject, and
the acquiring the personal information acquires the personal information of the subject based on the image of the subject captured by the camera.

5. The measuring apparatus as claimed in claim 1, further comprising:
a display device configured to display the first measured value and the second measured value.

6. The measuring apparatus as claimed in claim 1, wherein the processor performs the process further including
urging the subject to remain still by at least one of audio and display, until the first measured value satisfies the condition.

7. The measuring apparatus as claimed in claim 6, wherein the urging displays a message or an image through a one-way mirror film, or turns on a light source.

8. The measuring apparatus as claimed in claim 1, wherein the recording records the first measure value and the second measured value in correspondence with each other in the memory.

9. A measuring method comprising:
acquiring, by a processor, a first measured value from a first sensor that measures a weight of a subject;
judging, by the processor, whether the first measured value satisfies a condition in which a change in the first measured value within a predetermined time is a threshold value or less;
acquiring, by the processor, a second measured value from a second sensor that is other than the first sensor and detects biometric information of the subject other than the weight, in a case in which the judging judges that the first measured value satisfies the condition; and
recording, by the processor, the first measured value satisfying the condition and the second measured value in correspondence with each other in a memory.

10. The measuring method as claimed in claim 9, wherein the acquiring the second measured value acquires the second measured value from at least one of a distance measuring sensor that measures a height of the subject, a temperature sensor that measures a body temperature of the subject, and a camera that captures an image of the subject.

11. The measuring method as claimed in claim 9, further comprising:
acquiring, by the processor, personal information of the subject,
wherein the recording records the first measured value and the second measured value in the memory in correspondence with the personal information.

12. The measuring method as claimed in claim 11, wherein
the acquiring the second measured value acquires image data from a camera that captures an image of the subject, and
the acquiring the personal information acquires the personal information of the subject based on the image of the subject captured by the camera.

13. The measuring method as claimed in claim 9, further comprising:
displaying, by the processor, the first measured value and the second measured value on a display device.

14. The measuring method as claimed in claim 9, further comprising:
urging, by the processor, the subject to remain still by at least one of audio and display, until the first measured value satisfies the condition.

15. A non-transitory computer-readable storage medium having stored therein a program which, when executed by a computer, causes the computer to perform a process comprising:
acquiring a first measured value from a first sensor that measures a weight of a subject;
judging whether the first measured value satisfies a condition in which a change in the first measured value within a predetermined time is a threshold value or less;
acquiring a second measured value from a second sensor that is other than the first sensor and detects biometric information of the subject other than the weight, in a case in which the judging judges that the first measured value satisfies the condition; and
recording the first measured value satisfying the condition and the second measured value in correspondence with each other in a memory.

16. The non-transitory computer-readable storage medium as claimed in claim 15, wherein the acquiring the second measured value acquires the second measured value from at least one of a distance measuring sensor that measures a height of the subject, a temperature sensor that measures a body temperature of the subject, and a camera that captures an image of the subject.

17. The non-transitory computer-readable storage medium as claimed in claim 15, further comprising:
acquiring personal information of the subject,
wherein the recording records the first measured value and the second measured value in the memory in correspondence with the personal information.

18. The non-transitory computer-readable storage medium as claimed in claim 17, wherein
the acquiring the second measured value acquires image data from a camera that captures an image of the subject, and
the acquiring the personal information acquires the personal information of the subject based on the image of the subject captured by the camera.

19. The non-transitory computer-readable storage medium as claimed in claim 15, further comprising:
displaying the first measured value and the second measured value on a display device.

20. The non-transitory computer-readable storage medium as claimed in claim 15, further comprising:
urging the subject to remain still by at least one of audio and display, until the first measured value satisfies the condition.
